(19) United States
(12) Patent Application Publication
(10) Pub. No.: US 2023/0349875 A1
(43) Pub. Date: Nov. 2, 2023

(54) APPARATUS AND METHOD FOR MEASURING AIR QUALITY

(71) Applicant: uHoo PTE LTD, Singapore (SG)

(72) Inventors: Dustin Jefferson S. Onghanseng, Singapore (SG); Chun Lai Brain Lin, Tai Po (HK)

(21) Appl. No.: 18/027,823

(22) PCT Filed: Sep. 22, 2021
(86) PCT No.: PCT/SG2021/050573

§ 371 Date: Mar. 22, 2023

(30) Foreign Application Priority Data
Sep. 22, 2020 (SG) 10202009307Y

(51) Int. Cl. G01N 33/00; G01N 1/22; G01N 1/24

(52) U.S. Cl. CPC G01N 33/0031; G01N 1/2273; G01N 1/2205; G01N 33/007; G01N 33/0073; G01N 1/24; G01N 2001/245; G01N 2033/0072

(57) ABSTRACT

An air quality measurement unit comprising: a housing having an inlet for receiving an air flow and an outlet for venting said air flow; a plurality of sensors positioned in said housing forming an array of sensors, each sensor having a sensor inlet for receiving a portion of the air flow and a sensor outlet for venting said portion of air flow; each sensor arranged to measure an air quality parameter; wherein said sensors are placed such that the sensor inlet is positioned adjacent to the inlet.

### FIELD OF THE INVENTION

The invention relates to the measurement of air quality parameters. In particular, the invention relates to a unit having an array of sensors for measuring said parameters.

### BACKGROUND

Whilst commercial and industrial facilities may have complex air quality measuring systems, these are expensive capital items having dedicated information networks in order to meet industry standards and capable only to measure a limited number of air quality parameters, normally only up to three parameters. Other standalone industrial equipment measuring one specific air quality parameter are expensive capital items that are bulky/large in size and unable to provide long term trending and analysis in real time.

Air quality measuring devices for domestic use, however, are limited to providing a finite number of measurable parameters and are intrinsically basic in operation so as to only communicate said parameters directly to an observer.

### SUMMARY OF INVENTION

In a first aspect, the invention provides an air quality measurement unit comprising: a housing having an inlet for receiving an air flow and an outlet for venting said air flow; a plurality of sensors positioned in said housing forming an array of sensors, each sensor having a sensor inlet for receiving a portion of the air flow and a sensor outlet for venting said portion of air flow; each sensor arranged to measure an air quality parameter; wherein said sensors are placed such that the sensor inlet is positioned adjacent to the inlet.

In a second aspect, the invention provides an air quality measurement unit comprising: a housing having an inlet for receiving an air flow and an outlet for venting said air flow; said housing including a port arranged to removably receive a sensor module; the sensor module arranged to measure an air quality parameter; a control system arranged to interrogate said port to determine if a sensor module is present; wherein said control system further arranged to identify a type of the sensor module present, and receive data from it.

In a third aspect, the invention provides an air quality measurement system comprising: a remote data storage unit; at least one air quality measurement unit wirelessly connected to said remote data storage unit; wherein data collected by the at least one air quality measurement unit is arranged to be communicated to the remote data storage unit; wherein said received data is arranged to be calibrated by the remote data storage unit and a calibrated factor sent to the respective air quality measurement unit.

In a fourth aspect, the invention provides an air quality measurement unit comprising: a plurality of sensors, each sensor arranged to measure an air quality parameter; a control system to receive data from each sensor; said control system arranged to combine data from at least two sensors; wherein the control system is arranged to determine an index corresponding to said combined data.

In a fifth aspect, the invention provides a building control system comprising; a plurality of environmental control devices; said environmental control devices connected to a control system and arranged to receive control data from said control system; said control system arranged to receive sensor data from one or more sensors and determine an environmental index corresponding to said sensor data; wherein said control data comprises said environmental index, such that the environmental control devices adjust an environmental output as a function of said environmental index.

Thus, in one aspect, the unit is arranged so as to position sensors to maximize airflow as it enters the unit and so optimize the detection capability of the sensors. Importantly, a filter may be introduced to remove large particles of greater than 1 mm to prevent dust from clogging inside the sensor and main unit, and thus affecting the sensor accuracy.

Further, to drive air through the unit, a fan may be used to increase airflow and further the fan may be located proximate to the outlet to better utilize said airflow.

In a second aspect, the sensors may be provided in the form of sensor modules which are arranged to plug into and be removed from ports within the unit. A control system within the unit continuously may interrogate the ports to firstly detect whether a sensor module is connected and second to determine what type of sensor is engaged. Data received from the sensor is therefore directed to a processor by the control system with the control system identifying the type of data in order to communicate a result.

In a third aspect, the invention may comprise a system having a plurality of said units, all of which may be wirelessly connected to a centralized server such as a cloud arrangement. Data from each of the units may be communicated to the cloud, either having been processed within the unit or as raw data to be processed within the cloud. To this end, calibration of the sensors may occur within the unit or within the cloud. Calibration may be provided by comparing raw data having been received from a known sensor and comparing to a virtual calibration cell to calibrate the raw data according to any one or a number of criteria including the unit from which that data is collected, the location of the unit, and of course the type of sensor.

In a fourth aspect, the invention may provide for the control system either at a cloud level or at a unit level, an index to categorize an environmental condition. Such indexes may comprise two or more air quality parameters and so provide a more complex environmental condition. Said indexes may also be used to operate ancillary equipment arranged to reduce the impact of an elevated environmental condition represented by a respective index exceeding a predetermined threshold.

### BRIEF DESCRIPTION OF DRAWINGS

It will be convenient to further describe the present invention with respect to the accompanying drawings that illustrate possible arrangements of the invention. Other arrangements of the invention are possible and consequently, the particularity of the accompanying drawings is not to be understood as superseding the generality of the preceding description of the invention.

FIGS. 1A and 1B are various views of an air quality measurement unit according to one embodiment of the present invention;

FIG. 2 is a cross sectional view of the air quality unit of FIG. 1A;

FIG. 3A is a schematic view of an air quality measurement system according to one embodiment of the present invention;

FIG. 3B is a schematic view of an air quality measurement system according to a further embodiment of the present invention, ad;

FIG. 4 is a schematic view of an air quality measurement system and a pollutant cell according to a further embodiment of the present invention.

### DETAILED DESCRIPTION

The air quality unit according to the present invention is arranged for continuous monitoring of air quality. The deployment of the unit may vary upon the application, however for commercial use this would include a free standing unit or possible wall mounted. The air quality measurement unit is arranged to continually monitor air quality such as continuously taking readings from the airflow entering the unit.

It will be appreciated that the unit according to the various aspects of the invention may be adapted for indoor or outdoor use, whilst still falling within the broad invention. For instance, a unit for indoor use may be wall mounted, or self-standing for placement on a table, bench etc. The following description may generally refer to an indoor unit at various points, but this is not to be read as excluding embodiments encompassing outdoor use.

For instance, an adaptation for outdoor use, may also include a wall mounting or be free-standing. Further, an outdoor embodiment of the invention may allow the unit to be placed to optimize natural air flow, which may include being free-standing on the ground. It may also, or in the alternative, use a larger internal fan for increasing driven airflow. Still further, the unit may include, or adapted to work with, an airflow collector for channeling airflow into the inlet of the unit.

In one aspect of the invention the array of sensors includes selectively insertable sensor modules arranged to be inserted into ports within the unit. In a preferred embodiment the unit may be arranged to receive up to 15 selectively insertable sensor modules. The customized nature of the present invention is such that the unit may be re-configured by the operator by selecting different sensor modules, at different times, such as by inserting new sensor modules into available ports or removing unnecessary sensors and replacing these with new sensor modules.

In a further aspect, the unit may be connected to external nodes to communicate data and to also receive input, with said connection being wired or wirelessly. For instance, in one embodiment the unit may be wirelessly connected to a cloud for decentralized operation. That operation may include functions such as storing sensor readings, calibrating the sensor modules, processing raw data, etc.

The cloud may periodically update the control system resident in the unit. The control system may be arranged to operate the sensor modules and either process raw data or communicate the data to an external node such as a cloud. Further, the control system may be arranged to visually display processed sensor data either on a visual display on the unit or to a remote visual display.

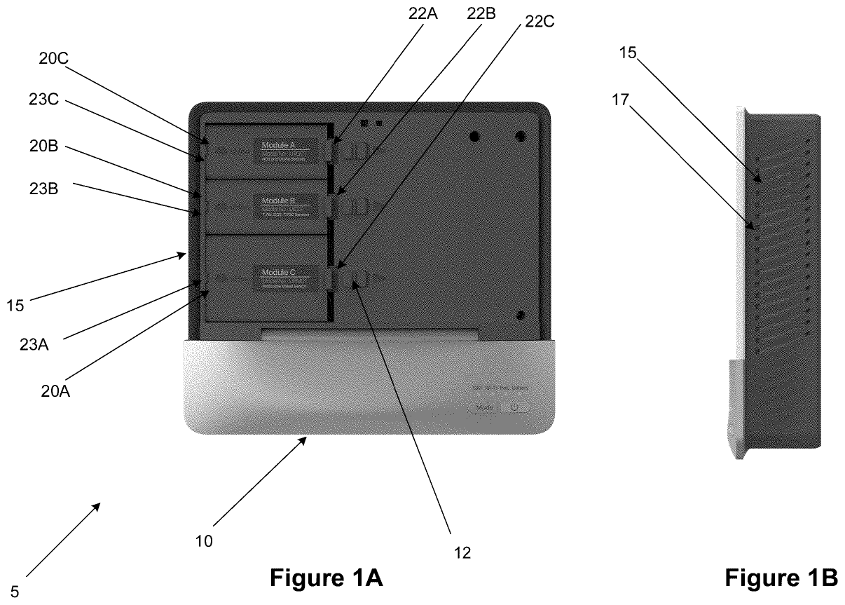

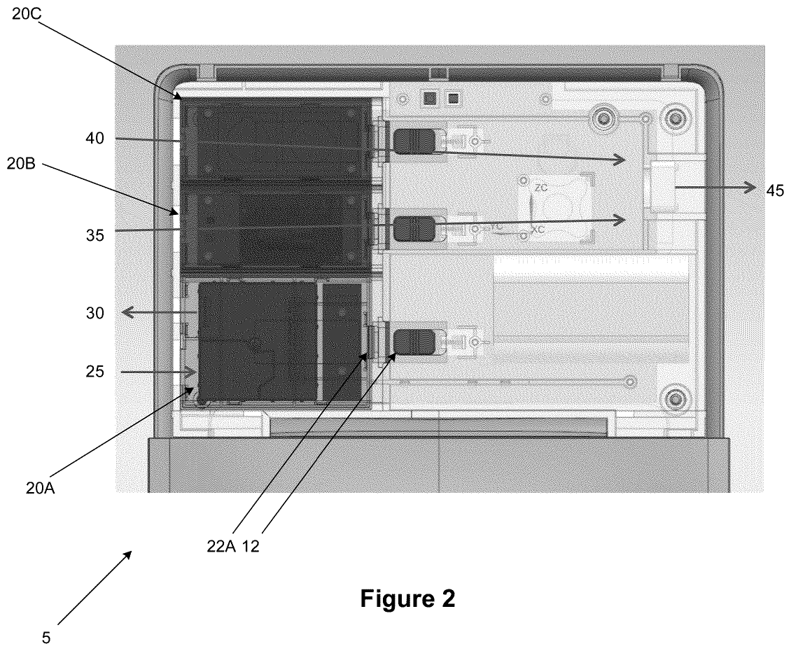

To this end, one embodiment of an air quality measurement unit 5 is shown in FIGS. 1A and 1B. Here the unit 5 includes a housing 10 having, in this case, 3 ports 22A to 22C. Inserted into these ports 22A to 22C are various sensor modules 20A to 20C which are positioned to directly receive an inflow from an inlet 15 into module inlets 23A to 23C. The inlet 15 includes a filter arranged to remove large particulate matter from the airflow. For instance, a filter 17 may be used to provide the device with an IP43 ingress protecting rating ensure that particular matter greater than 1 mm is prevented from entering the sensor array. It will be appreciated that the housing may have at least one non-removable sensor module, permanently operable within the housing. FIG. 2 shows a cross-sectional view of the unit 5 with the sensor modules 20A to 20C in place. In this embodiment, the installed sensor modules include a particulate matter sensor module 20A, a 5-in-1 sensor module 20B (Temperature+Humidity+Air Pressure+TVOC+CO₂) and an NO₂ and ozone sensor 20C. Each can be removed by engaging the spring-loaded latch 12 for sliding in and out of the respective port.

The various ports may vary such that, depending upon the type of sensor module required, that port may be dedicated for a particular type of module. For instance, in this embodiment port 22A is arranged for a sensor module that requires a separate inlet and outlet. Thus, as shown in FIG. 2, the particulate matter sensor module 20A is arranged to have a separate inflow 25 and outflow 30. This is separate from the other sensor ports having conventional inflow 35, 40, and outflow 45 passing through the sensor modules 20B, 20C. It is a particular advantage having the sensor modules arranged to receive air flow proximate to the inlet 15 and so optimizing the measurement characteristics.

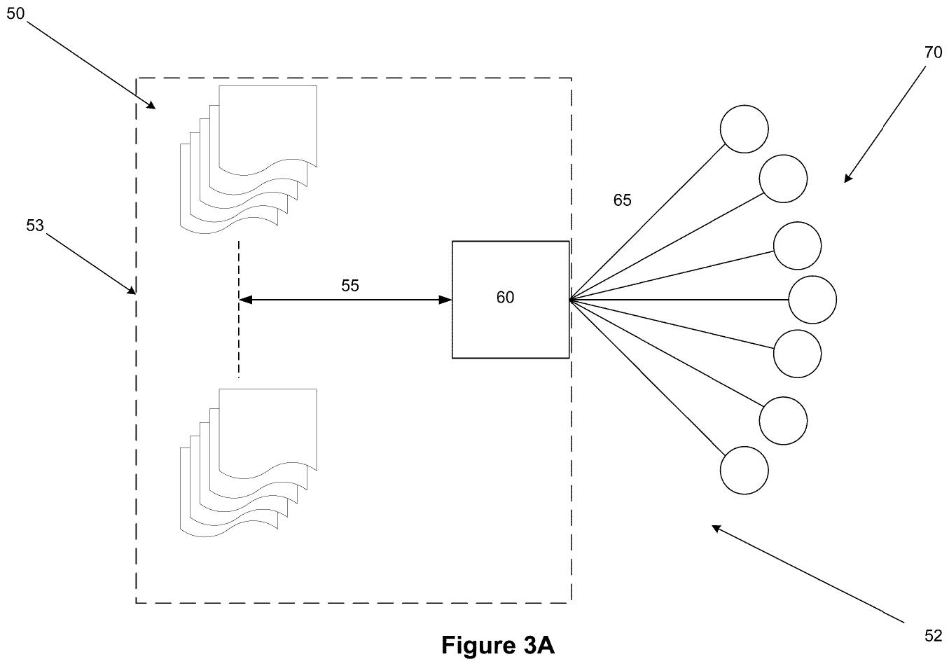

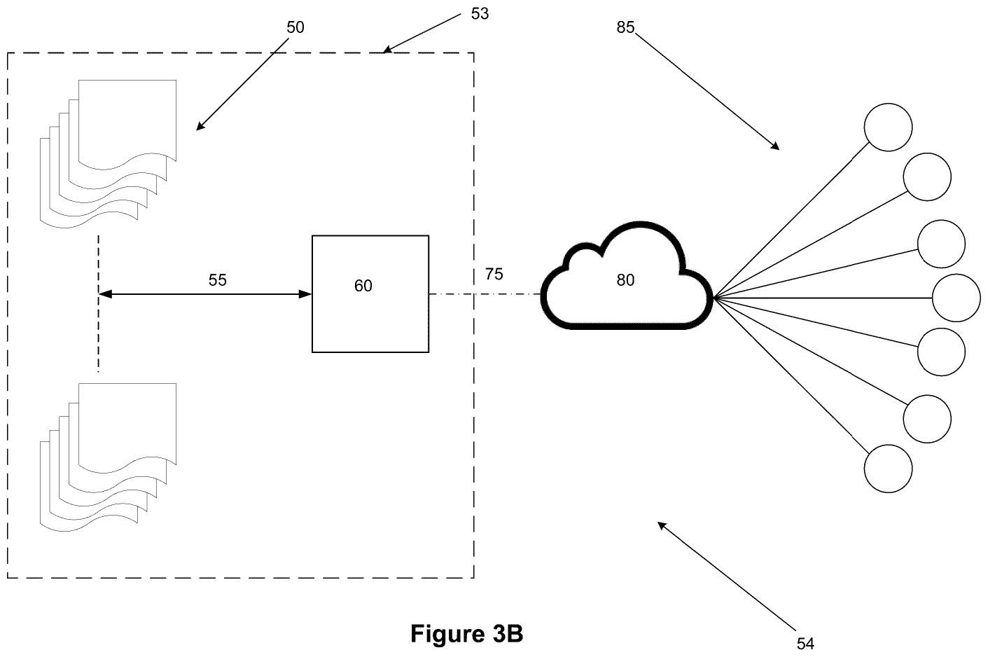

FIGS. 3A and 3B show various embodiments of the further aspect of the present invention. In a first embodiment of FIG. 3A, the system 52 comprises an array of sensors 50 which communicate data to a control system 60. The sensor array 50 and control system 60 reside within a unit 53 with the control system 60 of the unit 53 having either a wired or wireless connectivity capacity to communicate 65 data externally from the unit 53. In the embodiment of FIG. 3A, the data being communicated 65 is to an array of ancillary devices 70. In this embodiment, the control system having processed data from the sensor array 50, calculates an index for one or more environmental conditions. Using the index, control data is communicated 65 to various external devices in order to improve the environmental conditions in which unit 53 resides.

For example, the particulate matter sensor module may sense an increased concentration of 2.5 micron particles in the air. In an enclosed environment, the control system having detected the increase in 2.5 micron particles may operate a device such as an inlet for an air conditioning unit to close a louvre and thus reduce the inflow of air from outside. Alternatively, it may implement the use of a selectively operable filter to remove the particles before entering the environmental space.

In a further embodiment, the sensor array 50 may include a humidity sensor and a temperature sensor to calculate an index for sensible heat within the environment, based upon data from a temperature sensor and humidity sensor. By varying an air handling unit (AHU) system, the humidity and/or temperature may be reduced so as to reduce the sensible heat index within the comfort level required for said operation.

Thus, the system 52 according this embodiment may allow for the use of calculated indices to modify environmental conditions.

FIG. 3B shows a similar system 54 with a sensor array 50 communicating 55 data to a control system 60. However, in this embodiment, the control system 60 is capable of communicating 75 to a remote data storage unit, such as a cloud-based storage 80. The cloud-based storage 80 in this system 54 is then responsible for controlling the array of external devices 85 in a similar manner to that discussed with regard to the embodiment of FIG. 3A. The system 54 of FIG. 3B therefore has the capacity to receive input 75 from a plurality of units 53 to control, for instance, the environmental conditions in an office building whereby each individual office may have slightly different environmental conditions and therefore the cloud-based storage 80 receives a plurality of data to control a plurality of different external devices to provide the optimum environmental conditions for each of the individual offices.

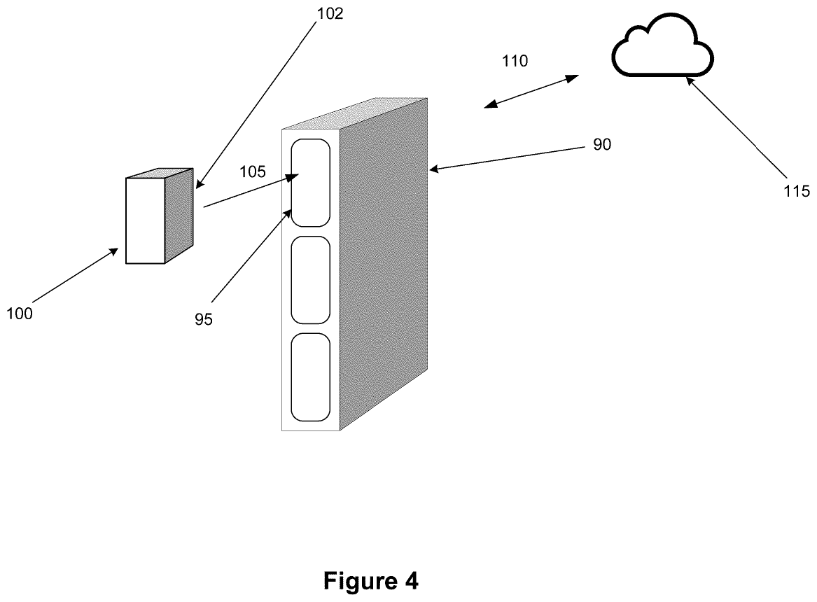

FIG. 4 shows a further embodiment whereby a test cell, or pollutant cell 100 is removably mountable 105. The test cell 100 may then be cooperatively mounted to an outlet 102 of the test cell 100 to an inlet 95 of an air quality measurement unit 90 according to the present invention. In this case, the unit 90 includes one or more sensor modules (not shown).

The test cell 100 may be formed by inserting a pollutant having a known concentration, with the pollutant corresponding to the sensor module to be tested or calibrated. The pollutant generator will vary with the type of pollutant, for instance for a gas pollutant (CO₂, NO₂ etc.) the pollutant generator may be a gas bottle connectable to a test cell inlet. For a particulate pollutant, the pollutant generator may be a container connectable to the same, or different test cell inlet, with the particulates fed into the test cell.

It will be appreciated that, for a particulate pollutant, to achieve the required concentration the entire test cell contents may require injecting into the test cell.

It will be further appreciated that for other pollutants, a variety of injection methods may be adopted, such as aerosol, gas injection of the pollutant gas, gas injection of an inert gas (such as nitrogen) to make the pollutant airborne during injection. To this end, the test cell may be specific to the pollutant.

In further embodiments, the test cell may contain several pollutants, directed to test or calibrate several sensor modules simultaneously.

The unit 90, or Device under Test (DUT), may be single- or multi-sensor module. Automated test equipment (ATE) may contain multiple test sockets that house each DUT. To this end, the DUT may be mounted within a test chamber and connected externally to a test/control system.

Reference equipment may be placed within the test chamber to provide reference measurement for the control system. Examples may include an NO₂ meter. Ozone meter, formaldehyde meter etc.

A control system embedded in the DUT may place the DUT into calibration mode. The control system receives the known pollutant concentration from the test cell, via any of the following non-limiting methods:
i) a QR code;
ii) RFID chip on the test cell to be read by an RFID reader in the unit;
iii) Manually entered by an operator, or;
iv) Other suitable means.

The unit commences initialization, with the control system determining the calibration from the collected raw data and the known pollutant concentration. From this, the control system determines a calibration factor for each sensor, then programs the calibration factor into the corresponding non-volatile memory on the sensor module.

The calibration factor may be in the form of a digital footprint representing the pollutants, and stored 110 on a cloud server 115. An operator could then commence the calibration mode, which would involve the unit downloading 110 the digital footprint from the cloud, and the control system either receiving the calibration factor or determining a calibration factor from the digital footprint.

The determination step may be necessary if the control system needs to adapt the data from the cloud, if the sensor modules within the unit are specific, and the digital footprint being more generalized.

1. An air quality measurement unit comprising:
a housing having an inlet for receiving an air flow and an outlet for venting said air flow;
a plurality of sensors positioned in said housing forming an array of sensors, each sensor having a sensor inlet for receiving a portion of the air flow and a sensor outlet for venting said portion of air flow;
each sensor arranged to measure an air quality parameter;
wherein said sensors are placed such that the sensor inlet is positioned adjacent to the inlet.

2. The air quality measurement unit according to claim 1, further including a fan positioned within said housing and adjacent the outlet, said fan arranged to draw said air flow into the inlet.

3. The air quality measurement unit according to claim 1, further including a filter placed on an inside face of the inlet, said filter arranged to block particles with the air flow greater than 1 mm.

4. The air quality measurement unit according to claim 1, wherein at least one sensor includes a sensor outlet adjacent to the inlet.

5. The air quality measurement unit according to claim 1, further including a visual indicator of the overall air quality.

6. An air quality measurement unit comprising:
a housing having an inlet for receiving an air flow and an outlet for venting said air flow;
said housing including at least one port arranged to removably receive a sensor module;
the sensor module arranged to measure an air quality parameter;
a control system arranged to interrogate said port to determine if a sensor module is present;
wherein said control system further arranged to identify a type of the sensor module present, to receive data from it.

7. The air quality measurement unit according to claim 6, wherein the control system is further arranged to detect a replaced sensor module.

8. The air quality measurement unit according to claim 6, wherein said control system is arranged to calibrate said received data;
said calibration based upon one or a combination of:
retrieving a calibration factor from a lookup table corresponding to the type of sensor module, comparing raw data to data from a calibration cell or receive calibration information from an external source.

9. The air quality measurement unit according to claim 6, wherein the at least one sensor module is arranged to measure more than one air quality parameter.

10. The air quality measurement unit according to claim 6, said housing includes a plurality of ports, each arranged to receive at least one sensor module.

11. The air quality measurement unit according to claim 6, said housing includes at least one non-removable sensor module.

12. The air quality measurement unit according to claim 6, further including a selectively removable test cell, the test cell containing a known concentration of a pollutant corresponding to the sensor module, said pollutant arranged to be injected into the unit and the control system arranged to receive raw data from the sensor module and determine a calibration factor for said sensor module, based upon said known pollutant concentration.

13. An air quality measurement system comprising:
a remote data storage unit;
at least one air quality measurement unit wirelessly connected to said remote data storage unit;
wherein data collected by the at least one air quality measurement unit is arranged to be communicated to the remote data storage unit;
wherein said received data is arranged to be calibrated by the remote data storage unit and a calibrated factor sent to the respective air quality measurement unit.

14. The air quality measurement system according to claim 13, wherein the remote data storage unit is arranged to use the calibrated data to operate equipment corresponding to the at least one air quality measurement unit to adjust environmental conditions.

15. The air quality measurement system according to claim 13, wherein said remote data storage unit is wirelessly connected to a plurality of air quality measurement units, said remote data storage unit arranged to calibrate and store received data from each air quality measurement unit.

16. The air quality measurement system according to claim 15, wherein said remote data storage unit is arranged to operate equipment corresponding to each air quality measurement unit.

17. An air quality measurement unit comprising:
a plurality of sensors, each sensor arranged to measure an air quality parameter;
a control system to receive data from each sensor;
said control system arranged to combine data from at least two sensors;
wherein the control system is arranged to determine an index corresponding to said combined data.

18. The air quality measurement system according to claim 17, wherein said control system is further arranged to compare said index to a threshold for said index corresponding to the air quality measurement unit;
such that the control system is arranged to operate external equipment to adjust environmental conditions to meet the corresponding index.

19. A building control system comprising:
a plurality of environmental control devices;
said environmental control devices connected to a control system and arranged to receive control data from said control system;
said control system arranged to receive sensor data from one or more sensors and determine an environmental index corresponding to said sensor data;
wherein said control data comprises said environmental index, such that the environmental control devices adjust an environmental output as a function of said environmental index.

20. The building control system according to claim 19, wherein the environmental devices are arranged to communicate the adjustment of the environmental output to said control system so as to adjust the index based upon any one or a combination of the sensor data and adjustment of the environmental output.
